(19) United States
(12) Patent Application Publication
(10) Pub. No.: US 2005/0227961 A1
(43) Pub. Date: Oct. 13, 2005

(54) Compositions and methods for treatment of neuropathic pain, fibromyalgia and chronic fatigue syndrome

(75) Inventors: Robert F. Kucharik, Glenmoore, PA (US); Brian T. Speicher, Lansdale, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Vela Pharmaceuticals, Inc., Ewing, NJ (US)

(21) Appl. No.: 10/999,737

(22) Filed: Nov. 30, 2004

Related U.S. Application Data
(60) Provisional application No. 60/560,816, filed on Apr. 8, 2004.

(51) Int. Cl. A61K 31/554; A61K 31/55; A61K 31/38; A61K 31/195

(52) U.S. Cl. 514/211.13; 514/217; 514/450; 514/431; 514/567

(57) ABSTRACT

Compounds according to the Formula 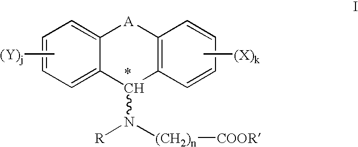 as defined herein are administered for the treatment of neuropathic pain, fibromyalgia and chronic fatigue syndrome.

### CROSS-REFERENCE TO RELATED APPLICATION

This application claims the benefit of copending U.S. Provisional Application Ser. No. 60/560,816, filed Apr. 8, 2004, the entire disclosure of which is incorporated herein by reference.

### FIELD OF THE INVENTION

The present invention relates to methods of treatment for neuropathic pain, fibromyalgia and chronic fatigue syndrome (CFS).

### BACKGROUND OF THE INVENTION

A. Tianeptine

Tianeptine, which has the systematic name 7-[(3-chloro-6,11-dihydro-6-methyl-dibenzo[c,f][1,2]thiazepin-11-yl)amino]heptanoic acid S,S-dioxide, is an antidepressant of the dibenzothiazepine type. Tianeptine is known to have psychostimulant, antidepressive, analgesic, antitussive, antihistaminic and gastric antisecretory properties. See, U.S. Pat. No. 3,758,528 of Malen et al., the entire disclosure of which is incorporated herein by reference. Tianeptine acts as a serotonin reuptake accelerator, in that it increases the presynaptic uptake of serotonin. A sodium salt of tianeptine is currently marketed over-the-counter in Europe under the trademark STABLON®. Tianeptine is used to treat neurotic or reactive states of depression, angiodepressive states with somatic complaints such as digestive problems, angiodepressive states observed in alcoholic detoxification, and asthma. The chemical structure of tianeptine is given below:

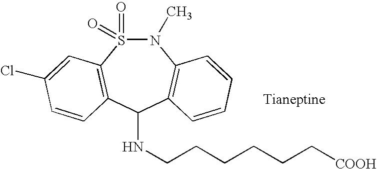

B. Tianeptine Contrasted With Tricyclic Antidepressants

Though tianeptine has been referred to in scientific literature as a tricyclic antidepressant (TCA), both the chemical structure and the biological mechanism of action of tianeptine are significantly different from that of classic TCAs. Shown below are the chemical structures of classic TCAs. The TCAs may be divided into secondary amines (desipramine, nortryptyline and protryptyline) and the tertiary amines (imipramine, amitriptyline and doxepin). The TCAs may also be characterized by whether the side chain is attached to the ring system via a single bond or by a double bond. However, all classic TCAs are characterized by an amino-(C₃)-alkyl sidechain, which is a significant departure from the carboxy-(C₆)-alkylamino sidechain of tianeptine.

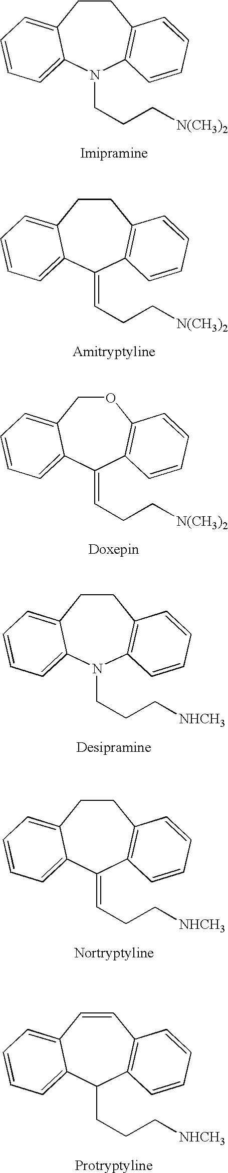

The biological activity of the classic TCAs is significantly different from that of tianeptine. The classic TCAs act by presynaptically inhibiting reuptake of serotonin and norepinephrine. See, The Merck Manual, 16^{th} Edition, Treatment of Unipolar and Bipolar Disorders, page 1603-1604, the entire disclosure of which is incorporated herein by reference. In contrast, tianeptine is a serotonin reuptake accelerator. See, Wilde et al., Clin. Neuropharmacol., 1998, 11, Suppl 2:S, page 74-82, the entire disclosure of which is incorporated herein by reference.

C. Known Tianeptine Analgesic Activity

Compounds according to Formula I, as defined herein, are disclosed by Malen et al. as having been tested for analgesic activity according to the “hot plate” test method of Woolfe et al., J. Pharmacol. Exp. Ther. 80, page 300-307, 1944, the entire disclosures of which are incorporated herein by reference.

According to the Woolf procedure, a heated platform is maintained at temperatures of 50 to 70° C. Rats are dropped onto the platform from an elevation of 5 cm and the latency to emit pain behaviors is recorded. The upper temperature limit of 70° C. is used to prevent tissue damage to the paws of animals. Each trial lasts 30 seconds, and the time interval, termed “latency,” for the pain behaviors to occur decreases as the temperature of the hot-plate increases. In these studies, longer latencies to elicit pain behaviors provide evidence of drug-induced analgesia.

The “hot plate” test elicits nociceptive pain. Nociceptive pain represents the healthy pain response to thermal and mechanical challenges that might result in tissue damage to an organism. The healthy nociceptive response requires proper functioning of an intact nervous system.

D. Neuropathic Pain

Neuropathic pain, in contrast to nociceptive pain, comprises a perturbation of pain signaling pathways resulting from electrophysiological instability which may be caused by injury to nerve tissue. See, Summer, Curr. Opin. Neurol., October, 2003, 16(5), page 623-628 and Krarup, Curr. Opin. Neurol., October, 2003, 16(5), page 603-612, the entire disclosures of which are incorporated herein by reference.

Central or peripheral nerve tissue damage may result in heightened sensitivity to non-noxious stimuli, and/or an exaggerated response to mild to moderately noxious stimuli. A simple focal peripheral nerve injury may initiate a range of peripheral and central nervous system processes that may contribute to persistent pain and abnormal sensation. See, Dworkin et al., Arch. Neurol., November, 2003; 60:1524-34, the entire disclosure of which is incorporated herein by reference.

The manifestation of neuropathic pain may comprise a number of positive and negative symptoms. See, Bonica's Management of Pain, 3rd Edition, ISBN 06833042623. Positive sensory phenomena relate to the exaggerated perception of stimuli (allodynia, hyperalgesia, hyperpathia), wherein application of modest stimuli causes the false perception of a disproportionately large stimuli. Positive motor symptoms include increased muscle tone, tremor, dystonia, and dyskinesia. Negative sensory phenomena include an inappropriate response to light touch, vibration, joint position, pin prick, or warm/cold application to the affected region. Negative motor symptoms include hypotonia, decreased muscle strength, and decreased endurance. The particular profile of positive and negative symptoms often corresponds to the specific insult to the nervous system.

Various medical conditions and external factors, including diabetes, i.e., diabetic neuropathy (DN), hypothyroidism, uremia, nutritional deficiencies, herpes zoster (shingles), alcoholism, stroke, HIV, multiple sclerosis, cancer and exposure to toxic substances, including chemotherapy (primarily chemotherapy with vincristine, cisplatin, zalcitabine, and paclitaxel). Other acquired and inherited disorders, including Guillain-Barre syndrome (GBS), postherpetic neuralgia (PHN), Charcot-Marie-Tooth (CMT) disease, complex regional pain syndrome type 1 (CRPS-1), ischemic neuropathy, painful spasticities, and other nervous system disorders that have pain as an attendant sign and/or symptom may also be associated with neuropathic pain. See, Carter et al., Physical Medicine and Rehabilitation Clinics of North America, 2001May; 12(2):447-59, the entire disclosure of which is incorporated herein by reference.

Models for neuropathic pain do not test pain response by a healthy nervous system. Rather, neuropathic pain models test the abnormal pain response resulting from damaged nerve tissue. One model produces neuropathic pain in test animals by surgically ligating spinal nerves. See, Chung et al., Pain, 50, p 355-363, 1992, the entire disclosure of which is incorporated herein by reference. The Chung et al. model provides a widely accepted model for peripheral neuropathic pain in humans. The Chung et al. model detects antihyperalgesic activity in rats suffering from neuropathic pain by employing a surgical procedure to form a spinal nerve ligature.

The spinal nerve ligature produces a constriction injury that serves to model the perturbations associated with peripheral neuropathic injury in a mammal. Specifically, the phenomena of thermal hyperalgesia, cold allodynia, and tactile allodynia manifest themselves. Subsequent to the surgery to create the constriction injury, the rats are challenged with thermal and mechanical stimuli to determine the degree of sensitivity. Demonstration of an ability to decrease the abnormal pain sensitivity effected by the spinal nerve ligature is predictive of an agent's potential efficacious treatment of neuropathic pain.

E. Fibromyalgia

Fibromyalgia is a syndrome which is a frequent cause of chronic, widespread pain and is estimated to affect 2-4% of the population. Fibromyalgia and CFS are thought to be related. See, Kranzler et al., U.S. Pat. No. 6,635,675, the entire disclosure of which is incorporated herein by reference. However, the symptom profile of fibromyalgia differs from that of CFS, ie., pain is the major symptom reported in fibromyalgia while fatigue is the major symptom reported in CFS.

Fibromyalgia is characterized by a generalized heightened perception of sensory stimuli. Patients with fibromyalgia display abnormalities in pain perception in the form of both allodynia (pain with innocuous stimulation) and hyperalgesia (increased sensitivity to painful stimuli). Clinically, fibromyalgia is characterized by general aches or stiffness, primarily musculoskeletal in origin, involving three or more anatomical sites for at least three months and at least six typical and reproducible tender points. Other associated symptoms of fibromyalgia include fatigue, nonrestorative sleep and memory difficulties.

Fibromyalgia is likely to be caused by dysfunction of various components of the central nervous system. See, Yunus, J. Rheumatol., 1993, 19, page 846-850, the entire disclosure of which is incorporated herein by reference. Evidence has accumulated that aberrant function of the autonomic nervous system, and in particular the sympathetic nervous system, is responsible for the symptoms of fibromyalgia.

Abnormal findings in fibromyalgia patients strongly indicate a neuropathic pain syndrome, reminiscent of complex regional pain syndrome or postherpetic neuralgia. In addition, fibromyalgia seems to share similar characteristics with these neuropathic pain syndromes, including ineffective response to many analgesics. See, Staud, Pain Med., September, 2(3), 208-15 (2001), the entire disclosure of which is incorporated herein by reference.

F. Chronic Fatigue Syndrome

CFS is a disorder characterized by fatigue of an incapacitating nature lasting at least six months. As stated above, CFS and fibromyalgia, though manifesting different symptom profiles, are believed to be related. CFS symptoms include, but are not limited to, mild fever or chills, sore throat, painful lymph nodes, unexplained general muscle weakness, myalgias, prolonged generalized fatigue after exercise previously tolerated, generalized headaches, migratory arthralgias, neuropsychotic complaints, sleep disturbance, and description of a main symptom complex developing over a few hours to a few days.

CFS diagnostic criteria have been established by the U.S. Centers for Disease Control and Prevention. The diagnostic criteria include medically unexplained fatigue of at least six months duration that is of new onset, not a result of ongoing exertion and not substantially alleviated by rest, and a substantial reduction in previous levels of activity. In addition, diagnosis of CFS involves the determination of the presence of four or more of the following symptoms: subjective memory impairment, tender lymph nodes, muscle pain, joint pain, headache, unrefreshing sleep, and postexertional malaise lasting more than 24 hours. See, Reid et al., 2000, British Medical Journal 320, page 292-296, the entire disclosure of which is incorporated herein by reference.

Fibromyalgia and CFS have been treated with the same medications. Some medications currently employed to treat CFS and/or fibromyalgia include, but are not limited to, analgesics, hypnotics, and immune suppressants. Though numerous agents are used to treat fibromyalgia and CFS patients, no single pharmacological agent or combination of agents has been demonstrated to be effective in the treatment of either.

Agents presently used to treat neuropathic pain, fibromyalgia and CFS are not always effective. Some may produce serious side effects. Some, such as opioid analgesics, may have serious addictive liability. There is a need for agents which are effective in treating neuropathic pain, fibromyalgia and CFS, as well as pain attendant to nervous system disorders. In particular, there is a need for agents with few side effects and low liability for addiction, that are appropriate for long-term use in treatment and prevention of these disorders.

### SUMMARY OF THE INVENTION

Compounds of Formula I can prevent or alleviate symptoms of neuropathic pain, fibromyalgia and CFS.

According to one embodiment of the invention, a method of treating neuropathic pain, fibromyalgia or CFS therefore comprises administering an effective amount of at least one compound according to Formula I, or a pharmaceutically acceptable salt thereof, to a subject in need of such treatment. Formula I is: 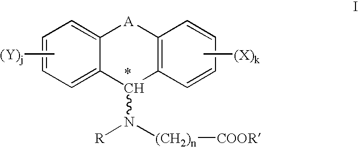
wherein:
A is diradical selected from the group consisting —(CH₂)ₘ—, —CH═CH—, —(CH₂)ₚO—, —(CH₂)ₚS—, —(CH₂)ₚSO₂—, —(CH₂)ₚNR₁— and —SO₂NR₂—; wherein:
   m is 1, 2 or 3; preferably 1 or 2;
   p is 1 or 2; preferably 1;
   R₁ is selected from the group consisting of hydrogen and C₁-C₅ alkyl; and
   R₂ is C₁-C₅ alkyl;
X and Y are independently selected from halogen;
j and k are integers independently selected from the group consisting of 0, 1 and 2; preferably 0 and 1;
R and R′ are independently selected from the group consisting of hydrogen and C₁-C₅ alkyl;
n is an integer from 1 to 12 inclusive, preferably from 2 to 10 inclusive, most preferably from 4 to 8 inclusive; and
* denotes an asymmetric carbon and the bond designated by [symbol] indicates that the absolute conformation about the asymmetric carbon may be either (R) or (S) when all four groups attached to the asymmetric carbon are nonequivalent.

According to one preferred embodiment, A is —SO₂—NR₂—, and/or j and k are 0.

The invention is also directed to the use of a compound according to Formula I, or a pharmaceutically acceptable salt thereof, for preparation of a medicament for treatment of neuropathic pain, fibromyalgia or CFS.

### DEFINITIONS

The term “alkyl”, by itself or as part of another substituent means a straight, branched or cyclic chain hydrocarbon radical, including di- and multi-radicals, having the number of carbon atoms designated (i.e. C₁-C₅ means one to five carbons). Straight chain alkyl groups are preferred. Examples of alkyl groups include: methyl, ethyl, propyl, isopropyl, butyl, isobutyl, tert-butyl, cyclopropylmethyl, pentyl, cyclopentyl and neopentyl.

The term “halogen” means iodine, fluorine, chlorine and bromine atoms. Preferred halogens are fluorine, chlorine and bromine atoms.

As used herein, “optically active” refers to a property whereby a material rotates the plane of plane-polarized light. A compound that is optically active has a chemical structure which is nonsuperimposable on its mirror image. As used herein, the property of nonsuperimposability of an object on its mirror image is called “chirality.” The most common structural feature producing chirality is an asymmetric carbon atom; i.e., a carbon atom having four nonequivalent groups attached thereto.

As used herein, “enantiomer” refers to each of the two nonsuperimposable isomers of a pure compound that is optically active. Single enantiomers are designated according to the Cahn-Ingold-Prelog system, which is a well-known set of priority rules for ranking the four groups attached to an asymmetric carbon. See, e.g., March, Advanced Organic Chemistry, 4^{th} Ed., (1992), p. 109, the entire disclosure of which is herein incorporated by reference. For example, once the priority ranking of the four groups attached to an asymmetric carbon of a molecule is determined, the molecule is oriented so that the lowest ranking group is pointed away from the viewer. If the descending rank order of the other groups proceeds clockwise, the molecule is designated (R). If the descending rank of the other groups proceeds counterclockwise, the molecule is designated (S). In the example below, the Cahn-Ingold-Prelog ranking sequence is A>B>C>D. The lowest ranking atom, D is oriented away from the viewer.

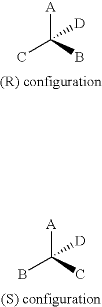

As used herein, “racemate” or “racemic compound” refers to a 50-50 mixture of two enantiomers of a compound such that the mixture does not rotate plane-polarized light.

By “(R)-enantiomer substantially free of the (S)-enantiomer” is meant a compound that comprises 80% or more by weight of the (R)-enantiomer, and likewise contains 20% or less by weight of the (S)-enantiomer as a contaminant. By “(S)-enantiomer substantially free of the (R)-enantiomer” is meant a compound that comprises 80% or more by weight of the (S)-enantiomer, and likewise contains 20% or less by weight of the (R)-enantiomer as a contaminant.

### DETAILED DESCRIPTION OF THE INVENTION

The compounds of Formula I may be readily prepared by known methods. For example, compounds of Formula I may be prepared according to Scheme 1, Step B by condensation of the halogenated derivative of Formula IB with an aliphatic ω-amino ester of Formula IC. The reaction of Step B of Scheme 1 is preferably carried out in a suitable solvent in the presence of a suitable acid scavenger. Suitable solvents include acetonitrile, dimethylformamide (DMF) and nitromethane. Suitable acid scavengers include tertiary amines, aromatic amines such as pyridine, and inorganic bases such as alkali metal and alkaline earth metal carbonates or bicarbonates. The acid scavenger may comprise an excess of the aliphatic ω-amino ester of Formula IB. Some suitable acid scavengers, such as triethyl amine or pyridine may also serve as the solvent for the reaction of Step B. Compounds according to Formula I that are esters may then be hydrolyzed to yield compounds of Formula I that are carboxylic acids. The hydrolysis reaction is preferably carried out in a suitable aqueous solvent using either an acid or a base. Suitable aqueous solvents include water and mixtures of water with at least one water-miscible organic solvent such as an aliphatic alcohol, acetonitrile, tetrahydrofuran or acetone.

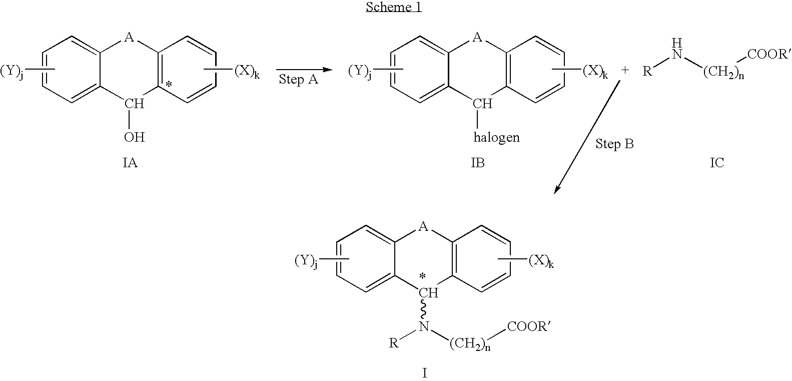

As shown in Scheme 1, Step A, halogenated derivatives of Formula IB may be prepared by reaction of a hydroxy derivative of Formula IA with a suitable halogenating reagent. The reaction of Step A is preferably carried out in a suitable solvent such as, for example, dichloromethane (DCM), chloroform, acetonitrile or THF. Suitable halogenating reagents include for example, dry HCL, and thionyl chloride.

Suitable synthetic methods are found, for example, in U.S. Pat. Nos. 4,766,114, 3,758,528 and 3,821,249, all of Malen et al., and U.S. Pat. No. 6,441,165 of Blanchard et al., the entire disclosures of which are herein incorporated by reference.

Certain compounds of Formula I, such as tianeptine (see, Formula II, below), possess an asymmetric carbon. The position of the asymmetric carbon is denoted by an asterisk (*) in Formula I. This carbon is asymmetric when four nonequivalent groups are attached to it. One skilled in the art can readily determine which compounds of Formula I possess an asymmetric carbon.

Those compounds of Formula I which have an asymmetric carbon at the position marked by an asterisk may exist as (R) and (S) enantiomers. Typically, the (R) and (S) enantiomers of a given compound of Formula I exist as a racemate. In the practice of the present invention, both racemates and individual (R) or (S) enantiomers of a compound of Formula I can be used to treat neuropathic pain, fibromyalgia or CFS. According to certain embodiments of the invention, an (R)-enantiomer of a compound of Formula I which is substantially free of the corresponding (S)-enantiomer, or an (S)-enantiomer of a compound of Formula I which is substantially free of the corresponding (R)-enantiomer, is used to treat neuropathic pain, fibromyalgia or CFS.

To isolate the individual (R)- and (S)-enantiomers of a compound of Formula I, the racemate of that compound must be resolved. This resolution may be achieved by converting a racemic compound of Formula I into a pair of diastereomers, for example by covalently bonding to an optically active moiety or by salt formation with an optically active base or acid. Either method provides a molecule with a second chiral center, thus generating a pair of diastereomers. The diastereomeric pair may then be separated by conventional methods, such as crystallization or chromatography.

For example, racemic compounds of Formula I may be converted to the (S)-dibenzoyltartaric acid salt, which is a diastereomeric mixture of (S,S) and (R,S) configurations. The pairs of diastereomers (R,S) and (S,S) possess different properties (e.g., differential solubilities) and may thereby be separated by conventional separation methods. Fractional crystallization of diastereomeric salts from a suitable solvent is one such separation method.

Racemic compounds of Formula I may be separated into enantiomers without diastereomer formation, for example, by differential absorption on a chiral stationary phase of a chromatography (e.g., HPLC) column. Preparative HPLC columns suitable for diastereomer separation are commercially available with a variety of packing materials to suit a broad range of separation applications. Stationary phases suitable for resolving racemic compounds of Formula I include:
(i) macrocyclic glycopeptides, such as silica-bonded vancomycin which contains 18 chiral centers surrounding three pockets or cavities;
(ii) chiral α₁-acid glycoprotein;
(iii) human serum albumin; and
(iv) cellobiohydrolase (CBH).

Chiral α₁-acid glycoprotein is a highly stable protein immobilized onto spherical silica particles that tolerates high concentrations of organic solvents, high and low pH, and high temperatures. Human serum albumin is especially suited for the resolution of weak and strong acids and zwitterionic and nonprotolytic compounds, but is also used to resolve basic compounds. CBH is a very stable enzyme that that is typically immobilized onto spherical silica particles for separating enantiomers of basic drugs from many compound classes.

Other chromatographic techniques suitable for resolving racemic compounds of Formula I include chiral chromatography using macrocyclic glycopeptide as a stationary phase on a Chirobiotic V™ column (ASTEAC, Whippany, N.J.) as described in U.S. Pat. No. 6,080,736, the entire disclosure of which is herein incorporated by reference, and chiral chromatography using a chiral α₁-acid glycoprotein as a stationary phase on a CHIRAL-AGP™ column (ChromTech, Cheshire, UK), as described in Fitos et al. (J. Chromatogr., 1995, 709:265, the entire disclosure of which is herein incorporated by reference.

A preferred compound of Formula I for use in the present methods is tianeptine, or a pharmaceutically acceptable salt thereof. The structure of tianeptine is given in Formula II: 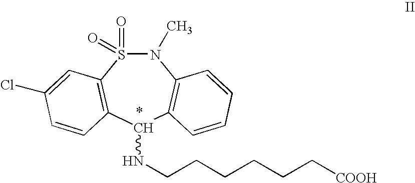
wherein:
* denotes an asymmetric carbon; and
the bond designated by 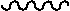
   indicates that the absolute conformation about the asymmetric carbon may be either (R) or (S).

Tianeptine may be readily obtained by one of ordinary skill in the art, for example by the synthetic techniques described above. Tianeptine is also sold commercially under the trademark STABLON®.

The (R) or (S) enantiomers of tianeptine may be isolated, for example, by the techniques discussed above. Thus, in preferred embodiments of the present invention, the (R)-enantiomer of tianeptine which is substantially free of the corresponding (S)-enantiomer, or the (S)-enantiomer of tianeptine which is substantially free of the corresponding (R)-enantiomer, is used in the present methods.

In the practice of the invention, the compounds of Formula I described above may take the form of a pharmaceutically-acceptable salt. The term “salts”, embraces salts commonly used to form alkali metal salts and to form addition salts of free acids or free bases.

For example, pharmaceutically-acceptable acid addition salts may be prepared from an inorganic acid or from an organic acid. Suitable inorganic acids include hydrochloric, hydrobromic, hydroiodic, nitric, carbonic, sulfuric and phosphoric acid. Suitable organic acids include aliphatic, cycloaliphatic, aromatic, araliphatic, heterocyclic, carboxylic and sulfonic classes of organic acids, such as formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, alginic, beta-hydroxybutyric, galactaric and galacturonic acid.

Suitable pharmaceutically acceptable base addition salts of the compounds of Formula I, include metallic salts made from calcium, magnesium, potassium, sodium and zinc, or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound of Formula I by reacting, for example, the appropriate acid or base with the compound of Formula I.

The compounds of Formula I, in particular tianeptine, can be used to treat neuropathic pain, fibromyalgia or CFS in a subject who has been diagnosed with either disorder. As used herein, a “subject” is includes humans and non-human mammals. Non-human mammals include bovines, ovines, porcines, equines, canines, felines, and rodents (e.g., rat, mouse, guinea pig and rabbit). Preferably, the subject is a human.

Treatment of neuropathic pain, fibromyalgia or CFS may refer to administration of at least one compound according to Formula I to a subject who has been diagnosed with such a disorder. Treatment may also refer to prophylactic administration to prevent neuropathic pain, fibromyalgia or CFS in a subject who is at risk of developing one or more of the disorders. Treatment also includes administration of a compound according to Formula I to a subject reporting one or more of the physiological symptoms of neuropathic pain, fibromyalgia or CFS, even when the diagnosis thereof has not yet been made.

In the practice of the invention, neuropathic pain, fibromyalgia and CFS are treated by administering an effective amount of at least one compound of Formula I to a subject in need of such treatment.

Treatment according to the present invention may comprise preventing, eradicating or ameliorating the neuropathic pain, fibromyalgia or CFS. Alternately, treatment according to the present invention may comprise preventing, eradicating or ameliorating one or more of the symptoms associated with neuropathic pain, fibromyalgia or CFS. For example, treatment of a patient suffering from neuropathic pain is accomplished not only when the underlying neuropathic pain is prevented, eradicated or ameliorated, but also when at least one symptom of the disorder is improved. Treatment may thus be accomplished, for example, when the patient reports decreased severity, duration, or recurrence of pain, a reduction in the number of anatomical sites affected by pain, or an improvement in abnormally heightened sensitivity to normally non-noxious stimuli.

Depression is often reported in subjects suffering from neuropathic pain, fibromyalgia or CFS, and has been characterized by some health care professionals as a symptom associated with these disorders. Tianeptine is known in the art to be useful as an antidepressant. Accordingly, treatment of a subject suffering from neuropathic pain, fibromyalgia or CFS with one or more compounds according to Formula I that causes an improvement solely in depression but not in at least one of the physiological symptoms associated with the disorder is neither contemplated by, nor considered effective for purposes of the present invention.

As used herein, an “effective amount” of a compound of Formula I used to treat fibromyalgia refers to the amount of the compound that prevents or alleviates one or more symptoms of fibromyalgia. Similarly, an “effective amount” of a compound of Formula I used to treat neuropathic pain refers to the amount of the compound that prevents or alleviates the neuropathic pain. Likewise, an “effective amount” of a compound of Formula I used to treat CFS refers to the amount of the compound that prevents or alleviates one or more symptoms of CFS. A physician can readily determine when symptoms of neuropathic pain, fibromyalgia or CFS are prevented or alleviated, for example through clinical observation of a subject, or through reporting of symptoms by the subject during the course of treatment.

One skilled in the art can readily determine an effective amount of a compound of Formula I to be administered, by taking into account factors such as the size, weight, age and sex of the subject, the extent of disease penetration or persistence and severity of symptoms, and the route of administration. Generally, an effective amount of the compounds of Formula I administered to a subject is from about 2 to about 100 mg per day, preferably from about 5 to about 60 mg per day, and more preferably about 30 mg per day. Higher or lower doses are also contemplated.

The compounds of Formula I may be administered to a subject by any route, for example by enteral (e.g., oral, rectal, intranasal, etc.) and parenteral administration. Parenteral administration includes, for example, intravenous, intramuscular, intraarterial, intraperitoneal, intravaginal, intravesical (e.g., into the bladder), intradermal, topical or subcutaneous administration. Also contemplated within the scope of the invention is the instillation of the compounds of Formula I into the body of the subject, for example in a controlled release formulation, with systemic or local release of the compound to occur over time or at a later time. According to some preferred embodiments, the compound of Formula I is localized in a depot for controlled release to the circulation or to a local site such as the gastrointestinal tract.

In the practice of the present methods, compounds of Formula I may be administered in the form of a pharmaceutical composition comprising at least one compound of Formula I and a pharmaceutically acceptable carrier. Pharmaceutical formulations of the invention may comprise from 0.1 to 99.99 weight percent of at least one compound of Formula I. The pharmaceutical compositions of the invention may be formulated according to standard practices in the field of pharmaceutical preparations. See, Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

By “pharmaceutically acceptable carrier” is meant any diluent or excipient that is compatible with the other ingredients of the formulation, and which is not deleterious to the recipient. The pharmaceutically acceptable carrier may be selected on the basis of the desired route of administration, in accordance with standard pharmaceutical practices.

Pharmaceutical compositions of the invention for parenteral administration may take the form of an aqueous or nonaqueous solution, dispersion, suspension or emulsion. In preparing pharmaceutical compositions of the invention for parenteral administration, at least one compound of Formula I may be mixed with a suitable pharmaceutically acceptable carrier such as water, oil (particularly a vegetable oil), ethanol, saline solutions (e.g., normal saline), aqueous dextrose (glucose) and related sugar solutions, glycerol, or glycols such as propylene glycol or polyethylene glycol. Pharmaceutical compositions of the invention for parenteral administration preferably contain a water-soluble salt of at least one compound of Formula I. Stabilizing agents, antioxidant agents and preservatives may also be added to the pharmaceutical compositions for parenteral administration. Suitable antioxidant agents include sulfite, ascorbic acid, citric acid or salts thereof, and ethylenediaminetetraacetic acid (EDTA) or a salt thereof. Suitable preservatives include benzalkonium chloride, methyl- or propyl-paraben, and chlorbutanol.

In preparing pharmaceutical compositions of the invention for oral administration, at least one compound of Formula I may be combined with one or more solid or liquid inactive ingredients to form tablets, capsules, pills, powders, granules or other suitable oral dosage forms. For example, at least one compound of Formula I may be combined with at least one pharmaceutically acceptable carrier such as a solvent, filler, binder, humectant, disintegrating agent, solution retarder, absorption accelerator, wetting agent absorbent or lubricating agent. In one embodiment, at least one compound of Formula I is combined with carboxymethylcellulose calcium, magnesium stearate, mannitol and starch, and is formed into tablets by conventional tableting methods. In a preferred embodiment, tianeptine is formulated into a tablet comprising cellulose and a calcium salt, as described in U.S. Pat. No. 5,888,542, the entire disclosure of which is herein incorporated by reference.

Pharmaceutical compositions of the invention may also be formulated so as to provide controlled-release of at least one compound of Formula I upon administration of the composition to a subject. Preferably, a controlled-release pharmaceutical composition of the invention is capable of releasing at least one compound of Formula I into a subject at a desired rate, so as to maintain a substantially constant pharmacological activity for a given period of time.

Formulation of controlled-release pharmaceutical compositions of the invention is within the skill in the art. Controlled release formulations suitable for use in the present invention are described in, for example, U.S. Pat. Nos. 5,674,533 (liquid dosage forms), U.S. Pat. No. 5,059,595 (gastro-resistant tablet), U.S. Pat. No. 5,591,767 (liquid reservoir transdermal patch), U.S. Pat. No. 5,120,548 (device comprising swellable polymers), U.S. Pat. No. 5,073,543 (ganglioside-liposome vehicle), U.S. Pat. No. 5,639,476 (stable solid formulation coated with a hydrophobic acrylic polymer), the entire disclosures of which are herein incorporated by reference.

Biodegradable microparticles may also be used to formulate controlled-release pharmaceutical compositions suitable for use in the present invention, for example as described in U.S. Pat. Nos. 5,354,566 and 5,733,566, the entire disclosures of which are herein incorporated by reference.

In one embodiment, controlled-release pharmaceutical compositions of the invention comprise at least one compound of Formula I and a controlled-release component. As used herein, a “controlled-release component” is a compound such as a polymer, polymer matrix, gel, permeable membrane, liposome and/or microsphere that induces the controlled-release of the compound of Formula I into the subject upon exposure to a certain physiological compound or condition. For example, the controlled-release component may be biodegradable, activated by exposure to a certain pH or temperature, by exposure to an aqueous environment, or by exposure to enzymes. An example of a controlled-release component which is activated by exposure to a certain temperature is a sol-gel. In this embodiment, at least one compound of Formula I is incorporated into a sol-gel matrix that is a solid at room temperature. This sol-gel matrix is implanted into a subject having a body temperature high enough to induce gel formation of the sol-gel matrix, thereby releasing the active ingredient into the subject.

The practice of the invention is illustrated by the following non-limiting example.

### EXAMPLE 1

### Neuropathic Pain—Thermal Stimulation Model in the Rat

A. Overview of the Neuropathic Pain Model

The effects of tianeptine on neuropathic pain were investigated by the method of Chung et al., Pain, 50, 355-363 (1992). The Chung et al. model detects antihyperalgesic activity in rats suffering from neuropathic pain by employing a surgical spinal nerve ligature and thermal stimulation to induce thermal hyperalgesia.

The nerve ligature produces a constriction injury that causes a distal perturbation in the pain signal pathways associated with the ipsilateral hind paw. Subsequent to the surgery, the rats are challenged with thermal stimuli to determine the degree of sensitivity in the target hindpaw. Test compounds that may be effective at treating neuropathic pain demonstrate an ability to decrease the pain sensitivity within the ipsilateral paw.

B. Animals and Test Substances

Forty male Rj: Wistar (Han) rats, 278-367 g body weight (weight at first experiment on testing day), were used in the study. The rats were divided into five treatment groups of 8 rats. The treatment groups received the following:
(a) Tianeptine, dispersed in carboxymethyl cellulose (CMC) (obtained from Coopération Pharmaceutique Francaise (CPF)) (1% in distilled water) at a dose of 3.0 mg/kg, P.O.;
(b) Tianeptine, as above, 10 mg/kg, P.O.;
(c) Tianeptine, as above, 30 mg/kg, P.O.;
(d) Morphine (obtained from CPF), dissolved in distilled water, at a dose of 128 mg/kg, P.O.; and
(e) Vehicle, 0.2% hydroxypropylmethylcellulose (HPMC) (obtained from CPF), dissolved in distilled water.

C. Procedure for the Surgical Phase of the Assay

The rats were anesthetized (sodium pentobarbital, obtained from Ceva Sante Animale, at a dose of 50 mg/kg i.p.). An incision at the L4-S2 level was performed to expose the left L5 and L6 spinal nerves. A ligature was tied tightly around each nerve. The wound was then sutured. The rats received an i.m. injection of 50 000 IU Penicillin G and were allowed to recover. Three weeks after the surgery, when the chronic neuropathic pain state was fully installed, rats were submitted to thermal stimulation of both the non-lesioned and the lesioned hindpaws.

D. Procedure for the Experimental Phase of the Assay

The experimental phase of the assay began three weeks after the surgery phase of the model was completed. The neuropathic pain assay comprised thermal stimulation of both the lesioned and unlesioned hindpaws of the rats. The experiment also included tactile stimulation of the rat hindpaws. However, the test compounds did not demonstrate activity versus tactile stimulation.

The neuropathic pain assay was performed as four separate experiments which assessed the pain response at 30 minutes, 1 hour, 2 hours and 4 hours following administration of the test substance. The four experiments were performed at 3, 4, 5 and 6 weeks following the surgical phase of the model. For each of the four experiments, each test substance was administered P.O., in a volume of 10 mL/kg of body weight.

The same rats were used for each of the four experiments. In each of the four experiments, prior to administration of a test substance, the rats were submitted to tactile stimulation and assigned to treatment groups matched on the basis of their pain response to tactile stimulation. The assignment of rats to treatment groups was done in this manner independently for each of the four experiments.

E. Apparatus and Procedure for Tactile Stimulation.

For tactile stimulation, the rat was placed under an inverted Plexiglas box (17×11×14 cm) on a grid floor. The tip of an electronic Von Frey probe (Model 1610 obtained from Bioseb BP 89 92370 Chaville, France) was then applied with increasing pressure to the non-lesioned and lesioned hindpaws. The force required to induce paw-withdrawal was automatically recorded. The tactile stimulation procedure was carried out 3 times for each paw. The mean force per paw was calculated to provide basic scores for each rat.

F. Apparatus and Procedure for Thermal Stimulation.

The apparatus employed for thermal stimulation consisted of 6 individual Plexiglas boxes (17×11×14 cm) placed upon an elevated glass floor (Model 7371, obtained from Ugo Basile Biological research apparatus Via G. Borghi 4321025 Comerio VA—Italy). For thermal stimulation, a rat was placed in the box and left free to habituate for 10 minutes. A mobile infrared radiant source was then focused under the non-lesioned and lesioned hindpaws. Paw-withdrawal by the rat interrupts the reflected radiation and switches off the counter and the light source, thereby automatically recording the paw-withdrawal latencies. In the event that no reaction was elicited, the test was terminated after 45 seconds to prevent tissue damage.

G. Results

The assay results are presented in Tables 1 and 2. The results represent the mean±the standard error of the mean (SEM) for each treatment group of eight rats. The data represent the percent change from the vehicle control. Statistical method and significance is indicated in Table 1. All statistical calculations were performed using commercial software (Microsoft Excel®, GB Stat® version 6.5), and verified test-by-test according to Porsolt & Partners Pharmacology's standardized internal procedures. All differences were considered statistically significant when the null hypothesis could be rejected at the risk α inferior to 0.05.

H. Conclusion

The data in Table 1 show that tianeptine produces a significant increase in paw withdrawal latency after thermal stimulation in the lesioned paw at 30 minutes at a dose of 30 mg/kg. This response is predictive of activity of the compound versus symptoms of neuropathic pain, fibromyalgia and CFS.

All references cited herein are incorporated by reference. The present invention may be embodied in other specific forms without departing from the spirit or essential attributes thereof and, accordingly, reference should be made to the appended claims, rather than to the foregoing specification, as indication the scope of the invention.

TABLE 1

| | Paw Withdrawal Latent (seconds) for Lesioned Paw | | | | | | | | | | | |
|---|---|---|---|---|---|---|---|---|---|---|---|---|
| Treatment | at 30 min | | | at 1 h | | | at 2 h | | | at 4 h | | |
| (mg/kg) | | p | | | | % | | | % | | | % |
| p.o. | mean ± SEM | value | % change | mean ± SEM | p value | change | mean ± SEM | p value | change | mean ± SEM | p value | change |
| Vehicle | 11.5 ± 3.9** | 0.0014 | −68% | 14.2 ± 3.6*** | 0.0005 | −61% | 17.9 ± 3.1* | 0.0148 | −50% | 19.7 ± 5.6** | 0.0024 | −55% |
| | (a) | | (a) | (a) | | (a) | (a) | | (a) | (a) | | (a) |
| Tianeptine | 12.7 ± 4.9 | 0.8506 | +10% | 20.3 ± 5.3 | 0.3599 | +43% | 20.6 ± 6.3 | 0.6979 | +15% | 13.8 ± 4.4 | 0.4215 | −30% |
| (3) | NS (b) | | (b) | NS (b) | | (b) | NS (b) | | (b) | NS (b) | | (b) |
| Tianeptine | 19.2 ± 6.0 | 0.3049 | +67% | 15.1 ± 4.5 | 0.8867 | +6% | 16.1 ± 5.1 | 0.7743 | −10% | 26.1 ± 5.4 | 0.4233 | +32% |
| (10) | NS (b) | | (b) | NS (b) | | (b) | (b) | | (b) | NS (b) | | (b) |
| Tianeptine | 33.3 ± 5.5** | 0.0064 | +190% | 29.0 ± 7.3 | 0.0932 | +104% | 16.9 ± 5.0 | 0.8724 | −6% | 19.3 ± 5.8 | 0.9552 | −2% |
| (30) | (b) | | (b) | NS (b) | | (b) | NS (b) | | (b) | NS (b) | | (b) |
| Morphine | 23.5 ± 6.4 | 0.1347 | +104% | 40.5 ± 3.0*** | 0.0001 | +185% | 16.7 ± 6.0 | 0.8637 | −7% | 21.7 ± 5.7 | 0.8090 | +10% |
| (128) | NS (b) | | (b) | (b) | | (b) | NS (b) | | (b) | NS (b) | | (b) |

(a) compared with non-lesioned paw (paired Student's t test): *= p < 0.5; **= p < 0.01; ***= p < 0.001
(b) compared with vehicle control (unpaired Student's t test): NS = Not Significant; **= p < 0.01; ***= p < 0.001

1. A method of treating a disorder selected from the group consisting of neuropathic pain, fibromyalgia and chronic fatigue syndrome in a subject in need of such treatment, comprising administering to the subject an effective amount of at least one compound of Formula I: 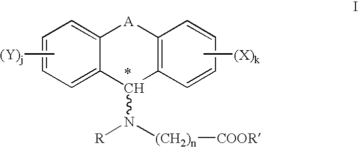
wherein:
A is diradical selected from the group consisting of —(CH₂)ₘ—, —CH═CH—, —(CH₂)ₚO—, —(CH₂)ₚS—, —(CH₂)ₚSO₂—, —(CH₂)ₚNR₁— and —SO₂NR₂—; wherein:
m is 1, 2 or 3;
p is 1 or 2;
R₁ is selected from the group consisting of hydrogen and C₁-C₅ alkyl; and
R₂ is C₁-C₅ alkyl;
X and Y are independently selected from halogen;
j and k are integers independently selected from the group consisting of 0, 1 and 2;
R and R′ are independently selected from the group consisting of hydrogen and C₁-C₅ alkyl;
n is an integer from 1 to 12 inclusive; and
* denotes an asymmetric carbon and the bond designated by [symbol] indicates that the absolute conformation about the asymmetric carbon may be either (R) or (S) when all four groups attached to the asymmetric carbon are nonequivalent.

2. A method of treating a disorder selected from the group consisting of neuropathic pain, fibromyalgia and chronic fatigue syndrome in a subject in need of such treatment, comprising administering to the subject an effective amount of at least one compound of Formula I: 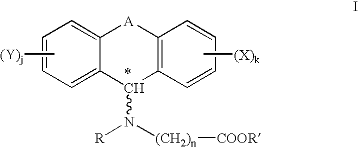
wherein:
A is diradical selected from the group consisting of —(CH₂)ₘ—, —CH═CH—, —(CH₂)ₚO—, —(CH₂)ₚS—, —(CH₂)ₚSO₂—, —(CH₂)ₚNR₁— and —SO₂NR₂—; wherein:
m is 1, 2 or 3;
p is 1 or 2;
R₁ is selected from the group consisting of hydrogen and C₁-C₅ alkyl; and
R₂ is C₁-C₅ alkyl;
X and Y are independently selected from halogen;
j and k are integers independently selected from the group consisting of 0 and 1;
R and R′ are independently selected from the group consisting of hydrogen and C₁-C₅ alkyl;
n is an integer from 1 to 12 inclusive; and
* denotes an asymmetric carbon and the bond designated by [symbol] indicates that the absolute conformation about the asymmetric carbon may be either (R) or (S) when all four groups attached to the asymmetric carbon are nonequivalent.

3. A method of treating a disorder selected from the group consisting of neuropathic pain, fibromyalgia and chronic fatigue syndrome in a subject in need of such treatment, comprising administering to the subject an effective amount of at least one compound of Formula I: 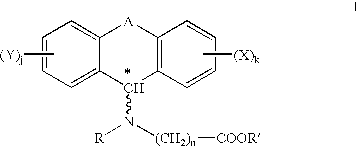
wherein:
A is diradical selected from the group consisting of —(CH₂)ₘ—, —CH═CH—, —(CH₂)ₚO—, —(CH₂)ₚS—, —(CH₂)ₚSO₂—, —(CH₂)ₚNR₁— and —SO₂NR₂—; wherein:
m is 1, 2 or 3;
p is 1 or 2;
R₁ is selected from the group consisting of hydrogen and C₁-C₅ alkyl; and
R₂ is C₁-C₅ alkyl;
X and Y are independently selected from halogen;
j and k are integers independently selected from the group consisting of 0 and 1;
R and R′ are independently selected from the group consisting of hydrogen and C₁-C₅ alkyl;
n is an integer from 4 to 8 inclusive; and
* denotes an asymmetric carbon and the bond designated by [symbol] indicates that the absolute conformation about the asymmetric carbon may be either (R) or (S) when all four groups attached to the asymmetric carbon are nonequivalent.

4. The method according to claim 1, wherein A is —SO₂NR₂—.

5. The method according to claim 4, wherein j and k are 0.

6. The method of claim 5, wherein the compound of Formula I is tianeptine or a pharmaceutically acceptable salt thereof.

7. The method of claim 6, wherein the compound is (R)-tianeptine, substantially free of the corresponding (S)-enantiomer.

8. The method of claim 6, wherein the compound is (S)-tianeptine, substantially free of the corresponding (R)-enantiomer.

9. The method of claim 1, wherein the subject is a human.

10. The method of claim 1, wherein the effective amount of the at least one compound of Formula I administered to the subject is from about 2 to about 100 mg per day.

11. The method of claim 1, wherein the effective amount of the at least one compound of Formula I administered to the subject is from about 5 to about 60 mg per day.

12. The method of claim 1, wherein the effective amount of the at least one compound of Formula I administered to the subject is about 30 mg per day.

13. The method of claim 1, wherein the at least one compound of Formula I is administered by an enteral administration route.

14. The method of claim 1, wherein the at least one compound of Formula I is administered by a parenteral administration route.

15. The method of claim 1, wherein the parenteral administration route is selected from the group consisting of intravenous, intramuscular, intraarterial, intraperitoneal, intravaginal, intravesical, intradermal, topical, subcutaneous, and instillation into the body of the subject.

16. The method of claim 1, wherein the disorder is neuropathic pain.

17. The method of claim 1, wherein the disorder is fibromyalgia.

18. The method of claim 1, wherein the disorder is chronic fatigue syndrome.

19. The method of claim 1, wherein the at least one compound of Formula I administered to the subject is contained in a pharmaceutical composition.

20. The method of claim 19, wherein the pharmaceutical composition comprises a controlled-release pharmaceutical composition.
